(19) United States
(12) Patent Application Publication
(10) Pub. No.: US 2018/0207175 A1
(43) Pub. Date: Jul. 26, 2018

(54) COMPOSITION FOR LOWERING INTRAOCULAR PRESSURE

(71) Applicant: HON HAI PRECISION INDUSTRY CO., LTD., New Taipei (TW)

(72) Inventor: HSIU-WEN CHIEN, New Taipei (TW)

(21) Appl. No.: 15/498,513

(22) Filed: Apr. 27, 2017

(30) Foreign Application Priority Data
Jan. 20, 2017 (CN) 201710048847.0

(51) Int. Cl. A61K 31/5578; A61K 31/5377; A61K 47/38; A61K 47/34; A61K 47/22; A61K 47/02; A61K 9/00

(52) U.S. Cl. CPC A61K 31/5578; A61K 31/5377; A61K 47/38; A61K 9/0048; A61K 47/22; A61K 47/02; A61K 47/34

(57) ABSTRACT

A tears-resistant and enduring intraocular pressure lowing composition comprises an intraocular pressure lowering drug, a mucoadhesive polymer, a PH sensitive polymer, and a solvent. When a PH is less than or equal to 7.03, the intraocular pressure lowing composition is in a liquid form, when the PH is greater than 7.03, the intraocular pressure lowing composition is in a gel form. The gel form of intraocular pressure lowering composition is not easily washed out by tears, so when the intraocular pressure of an eye is normal, the intraocular pressure lowering composition dropped in the eye is in gel form, thereby the intraocular pressure lowering composition can be retained in the eye for a long time.

### FIELD

The subject matter generally relates to an intraocular pressure lowering composition.

### BACKGROUND

Aqueous humor circulation in the eye is closely related to intraocular pressure, and the hindrance of the aqueous humor circulation has a considerable effect on the intraocular pressure. In particular, when the aqueous humor circulation is hindered, the intraocular pressure is increased, which may cause an ocular disease considered to be associated with aqueous humor circulation or intraocular pressure such as glaucoma or ocular hypertension. An intraocular pressure lowering composition is configured to lower intraocular pressure. As the intraocular pressure lowering composition can be easily washed out by tears, intraocular pressure lowering composition cannot remain in the eye for a long time.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein.

Several definitions that apply throughout this disclosure will now be presented.

The term “comprising” when utilized, means “including, but not necessarily limited to”; it specifically indicates open-ended inclusion or membership in the so-described combination, group, series, and the like. The term “about” when utilized, means “not only includes the numerical value, but also includes numbers closest to the numerical value”.

An exemplary embodiment of an intraocular pressure lowing composition comprises an intraocular pressure lowering drug, a mucoadhesive polymer, a PH sensitive polymer, and a solvent.

The intraocular pressure lowering drug has a mass percentage of about 0.001% to about 10% of the total mass of the intraocular pressure lowering composition. The mucoadhesive polymer has a mass percentage of about 0.03% to about 8.9% of the total mass of the intraocular pressure lowering composition. The PH sensitive polymer has a mass percentage of about 0.05% to about 15% of the total mass of the intraocular pressure lowering composition. The solvent has a mass percentage of about 56% to about 99.1% of the total mass of the intraocular pressure lowering composition.

In at least one exemplary embodiment, the intraocular pressure lowering composition has a PH value of about 6.5.

The intraocular pressure lowering drug may be selected from Timolol, Carteolol, Brimonidine, Pilocarpine, Dorzolamide, Brinzolamide, Acetazolamide, Latanoprost, Travoprost, Bimatoprost, or any combination thereof. The intraocular pressure lowering drug may be in granular, powder, or liquid form. The intraocular pressure lowering drug is configured to lower intraocular pressure and to prevent an ocular disease such as glaucoma or ocular hypertension caused by increased intraocular pressure.

The mucoadhesive polymer may be selected from methyl hydroxyethyl cellulose (MHEC), hydroxyethyl cellulose (HEC), hydroxypropyl methylcellulose (HPMC), sodium carboxymethyl cellulose (NaCMC), methylcellulose (MC), or any combination thereof. The mucoadhesive polymer increases a binding strength between the intraocular pressure lowing composition and an ocular mucosa.

The PH sensitive polymer comprises a plurality of polymer chains. When PH is less than or equal to 7.03, the plurality of polymer chains is a swelling structure, in other words, the PH sensitive polymer is a swelling structure. When PH is greater than 7.03, the plurality of polymer chains is an aggregate structure, in other words, the PH sensitive polymer is an aggregate structure. Thereby, when the PH of the intraocular pressure lowing composition is less than or equal to 7.03, the intraocular pressure lowing composition is in a liquid form, and when the PH of the intraocular pressure lowing composition is greater than 7.03, the intraocular pressure lowing composition is in a gel form.

The PH sensitive polymer may be selected from polysulfobetaine methacrylate, (poly SBMA), poly[(2-methyl-2-propene-1-sulfonic acid)]-co-[(3-acrylamidopropyl)trimethylammonium], poly(sodium 4-styrenesulfonate)-co-[(3-acrylamidopropyl)trimethylammonium], poly(2-acrylamido-2-methyl-1-propanesulfonic acid-co-[(3-acrylamidopropyl)trimethylammonium]), or any combination thereof.

The solvent may be water, sodium chloride (NaCl) solution, or a buffer solution. The buffer solution may be phosphate buffer, or 2-(N-Morpholino)ethanesulfonic acid monohydrate buffer (MES buffer). In at least one exemplary embodiment, the sodium chloride solution has a mass concentration of about 0.9%.

Under normal circumstances, the normal range of intraocular pressure is about 12 mmHg to about 22 mmHg, a PH value of an aqueous humor is 7.4. When the intraocular pressure is greater than or equal to 70 mmHg, the PH value of the aqueous humor is greater than or equal to 7.03 and less than 7.4, maybe even less than 7.03. If the intraocular pressure is consistently greater than 70 mmHg for less than 10 minutes, and then lowered to the normal range, the PH value aqueous humor can automatically recover to 7.4.

As the PH value of the intraocular pressure lowing composition is 6.5, the PH sensitive polymer swells. The PH sensitive polymer and the intraocular pressure lowering drug are evenly mixed, the intraocular pressure lowering drug can freely shuttle among the polymer chains, and the intraocular pressure lowering composition is in a liquid form. When the intraocular pressure lowing composition is applied in an eye with a normal intraocular pressure, as the PH value of the aqueous humor in the eye is 7.4, the PH sensitive polymer will reunite to form an aggregate structure. As the intraocular pressure lowering composition changes to a gel form from the liquid form, the intraocular pressure lowering drug is dispersed in the gel while not released from the gel to get into the aqueous humor. When the intraocular pressure is greater than or equal to 70 mmHg, the PH value will decrease and tend to approach 7.03 or lower. The PH sensitive polymer in the gel intraocular pressure lowering composition will swell, and the intraocular pressure lowering drug dispersed in the gel will be released from the gel and get into the aqueous humor to lower the intraocular pressure.

The liquid intraocular pressure lowering composition can be washed out easily by tears, while the gel intraocular pressure lowering composition is not easily washed out by tears. So when the intraocular pressure of an eye is normal, the intraocular pressure lowering composition in the eye is in gel form, thereby the intraocular pressure lowering composition can be retained in the eye for a long time.

When the intraocular pressure lowering composition is applied to an eye with a high intraocular pressure greater than or equal to 70 mmHg, the intraocular pressure lowering composition will not be changed to gel, and the intraocular pressure lowering drug will be released out and get into the aqueous humor, to lower the intraocular pressure. When the intraocular pressure recovers to normal, if the intraocular pressure lowering composition has not been completely consumed, the intraocular pressure lowering composition will change to a gel form and stay in the eye. When the intraocular pressure increases again, the PH sensitive polymer in the gel intraocular pressure lowering composition will again swell, and the intraocular pressure lowering drug dispersed in the gel will be released out and get into the aqueous humor, to lower the intraocular pressure.

The intraocular pressure lowering composition further comprises an anthocyanin. The anthocyanin has a mass percentage of about 0.001% to about 11% of the total mass of the intraocular pressure lowering composition.

It is well known that anthocyanin is a derivative of benzopyran. The anthocyanin is a water soluble flavonoids pigment, and the anthocyanin has antioxidant properties. Anthocyanins are widely found in most terrestrial plants, such as cactus, beet, onion, kidney bean, pomegranate, and grape, and anthocyanins provide a color for these plants. The anthocyanin can change colors depending on the PH of solution: the anthocyanin are pink in acidic solutions (pH<7), purple in neutral solutions (pH=7), greenish-yellow in alkaline solutions (pH>7), and colorless in strong alkaline solutions. The intraocular pressure lowering composition comprising the anthocyanin is pink. When the intraocular pressure lowering composition is dropped into an eye with a normal intraocular pressure, the intraocular pressure lowering composition comprising the anthocyanin changes to greenish-yellow. When the intraocular pressure increases, the PH value of the aqueous humor decreases, and the intraocular pressure lowering composition comprising the anthocyanin changes to purple or pink. Thereby the anthocyanin in the intraocular pressure lowering composition can indicate intraocular pressure to the user. When the color in the eye disappears, it signifies that the intraocular pressure lowering composition has been completely consumed, and there is a need for repeated application of intraocular pressure lowering composition.

### Example 1

The intraocular pressure lowering composition comprised Timolol, MHEC, polySBMA, anthocyanin, and sodium chloride solution. The sodium chloride solution had a mass concentration of 0.9%.

The Timolol had a mass percentage of 0.05% of the total mass of the intraocular pressure lowering composition, the MHEC had a mass percentage of 1.236% of the total mass of the intraocular pressure lowering composition, and the polySBMA had a mass percentage of 1.13% of the total mass of the intraocular pressure lowering composition. The anthocyanin had a mass percentage of 0.09% of the total mass of the intraocular pressure lowering composition, and the sodium chloride solution had a mass percentage of 97.494% of the total mass of the intraocular pressure lowering composition.

### Example 2

The intraocular pressure lowering composition comprised Timolol, MHEC, polySBMA, anthocyanin, and phosphate buffer.

The Timolol had a mass percentage of 0.108% of the total mass of the intraocular pressure lowering composition, the MHEC had a mass percentage of 0.73% of the total mass of the intraocular pressure lowering composition, and the polySBMA had a mass percentage of 1.052% of the total mass of the intraocular pressure lowering composition. The anthocyanin had a mass percentage of 0.048% of the total mass of the intraocular pressure lowering composition, and the phosphate buffer had a mass percentage of 98.062% of the total mass of the intraocular pressure lowering composition.

### Example 3

The intraocular pressure lowering composition comprised Timolol, HPMC, polySBMA, anthocyanin, and phosphate buffer.

The Timolol had a mass percentage of 0.98% of the total mass of the intraocular pressure lowering composition, the HPMC had a mass percentage of 0.82% of the total mass of the intraocular pressure lowering composition, and the polySBMA had a mass percentage of 0.932% of the total mass of the intraocular pressure lowering composition. The anthocyanin had a mass percentage of 0.068% of the total mass of the intraocular pressure lowering composition, and the phosphate buffer had a mass percentage of 97.2% of the total mass of the intraocular pressure lowering composition.

### Example 4

The intraocular pressure lowering composition comprised Timolol, HPMC, polySBMA, anthocyanin, and MES buffer.

The Timolol had a mass percentage of 0.98% of the total mass of the intraocular pressure lowering composition, the HPMC had a mass percentage of 0.59% of the total mass of the intraocular pressure lowering composition, and the polySBMA had a mass percentage of 0.619% of the total mass of the intraocular pressure lowering composition. The anthocyanin had a mass percentage of 0.072% of the total mass of the intraocular pressure lowering composition, and the MES buffer had a mass percentage of 97.739% of the total mass of the intraocular pressure lowering composition.

### Example 5

The intraocular pressure lowering composition comprised Latanoprost, MHEC, polySBMA, anthocyanin, and MES buffer.

The Latanoprost had a mass percentage of 1.36% of the total mass of the intraocular pressure lowering composition, the MHEC had a mass percentage of 0.96% of the total mass of the intraocular pressure lowering composition, and the polySBMA had a mass percentage of 0.892% of the total mass of the intraocular pressure lowering composition. The anthocyanin had a mass percentage of 0.092% of the total mass of the intraocular pressure lowering composition, and the MES buffer had a mass percentage of 96.696% of the total mass of the intraocular pressure lowering composition.

The embodiments shown and described above are only examples. Even though numerous characteristics and advantages of the present technology have been set forth in the foregoing description, together with details of the structures and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims.

What is claimed is:

1. An intraocular pressure lowing composition comprising:
an intraocular pressure lowering drug;
a PH sensitive polymer; and
a solvent.

2. The intraocular pressure lowing composition of claim 1, wherein the intraocular pressure lowering drug has a mass percentage of about 0.001% to about 10% of the total mass of the intraocular pressure lowering composition, the PH sensitive polymer has a mass percentage of about 0.05% to about 15% of the total mass of the intraocular pressure lowering composition, and the solvent has a mass percentage of about 56% to about 99.1% of the total mass of the intraocular pressure lowering composition.

3. The intraocular pressure lowing composition of claim 1, wherein the intraocular pressure lowering drug is selected from a group consisting of Timolol, Carteolol, Brimonidine, Pilocarpine, Dorzolamide, Brinzolamide, Acetazolamide, Latanoprost, Travoprost, Bimatoprost, and any combination thereof.

4. The intraocular pressure lowing composition of claim 1, wherein the PH sensitive polymer comprises a plurality of polymer chains.

5. The intraocular pressure lowing composition of claim 4, wherein when a PH of the intraocular pressure lowing composition is less than or equal to 7.03, the plurality of polymer chains is a swelling structure, when the PH of the intraocular pressure lowing composition is greater than 7.03, the plurality of polymer chains is an aggregate structure.

6. The intraocular pressure lowing composition of claim 1, wherein when a PH of the intraocular pressure lowing composition is less than or equal to 7.03, the intraocular pressure lowing composition is in a liquid form, when the PH of the intraocular pressure lowing composition is greater than 7.03, the intraocular pressure lowing composition is in a gel form.

7. The intraocular pressure lowing composition of claim 1, wherein the PH sensitive polymer is selected from a group consisting of polysulfobetaine methacrylate, (polySBMA), poly[(2-methyl-2-propene-1-sulfonic acid)]-co-[(3-acrylamidopropyl)trimethylammonium], poly(sodium 4-styrenesulfonate)-co-[(3-acrylamidopropyl)trimethylammonium], poly(2-acrylamido-2-methyl-l-propanesulfonic acid-co-[(3-acrylamidopropyl)trimethylammonium]), and any combination thereof.

8. The intraocular pressure lowing composition of claim 1, wherein the solvent is water, sodium chloride solution, or a buffer solution.

9. The intraocular pressure lowing composition of claim 1, wherein the intraocular pressure lowing composition further comprises a mucoadhesive polymer.

10. The intraocular pressure lowing composition of claim 9, wherein the mucoadhesive polymer has a mass percentage of about 0.03% to about 8.9% of the total mass of the intraocular pressure lowering composition.

11. The intraocular pressure lowing composition of claim 9, wherein the mucoadhesive polymer is selected from a group consisting of methyl hydroxyethyl cellulose, hydroxyethyl cellulose, hydroxypropyl methylcellulose, sodium carboxymethyl cellulose, methylcellulose, and any combination thereof.

12. The intraocular pressure lowing composition of claim 1, wherein the intraocular pressure lowing composition further comprises an anthocyanin.

13. The intraocular pressure lowing composition of claim 12, wherein the anthocyanin has a mass percentage of about 0.001% to about 11% of the total mass of the intraocular pressure lowering composition.
